(19) United States
(12) Patent Application Publication
(10) Pub. No.: US 2012/0078157 A1
(43) Pub. Date: Mar. 29, 2012

(54) Device For Providing Therapeutic Pressure To a Treatment Site

(75) Inventors: Sundaram Ravikumar, Briar Cliff Manor, NY (US); Guy Osborne, Trumbull, CT (US)

(73) Assignee: Sun Scientific, Inc., Dobbs Ferry, NY (US)

(21) Appl. No.: 12/891,762

(22) Filed: Sep. 27, 2010

(51) Int. Cl. A61F 13/00; A61B 17/00

(52) U.S. Cl. 602/79; 606/201; 602/43

(57) ABSTRACT

The subject invention includes devices for providing therapeutic pressure to a treatment site. The devices include a bladder portion and a dressing portion. The bladder portion has opposed upper and lower exterior walls sealed to one another along common peripheral edges thereof to form an interior pressure chamber therebetween, without a lip or flange extending around the perimeter of the peripheral edge. The dressing portion is integrated with the bladder portion or detachably provided so that none of the bladder material comes into contact with the patient's skin at the treatment site. The interior pressure chamber of the bladder portion may have a number of configurations and serves as a bolster for the dressing portion, used to aid in the healing process of the wound.

### BACKGROUND OF THE INVENTION

I. Field of the Invention

The subject invention relates to medical devices for providing therapeutic treatment pressure to a treatment site. More particularly, this invention relates to devices for applying pressure to wounds such as venous ulcers on a patient's leg having a dressing portion and a bladder portion. While the particular description herein relates to the treatment of wounds of the leg, the subject invention may be utilized to treat other related medical conditions requiring compression.

II. Background of the Related Art

Wounds on the lower leg are often caused by problem with blood circulation and degradation of veins and arteries. A venous ulcer, for example, is damage and loss of skin above the ankle that is the result of a problem with the veins in the leg. Venous ulcers typically develop on either side of the lower leg, above the ankle and below the calf. They are difficult to heal and often recur. Deep vein thrombosis is another medical condition that results in complete or partial obstruction of a deep vein. In some cases, unrelieved pressure produced by the calf muscle pump on the perforator valves may cause these valves to become incompetent. When this occurs, there will be a large rise in the pressure in the superficial system, which may force proteins and red cells out of the capillaries and into the surrounding tissue. Here, the red cells break down releasing a red pigment that causes staining of the skin, an early indicator of possible ulcer formation. Venous leg ulcers are generally shallow and red in color. The skin surrounding the ulcer is frequently discolored due to the staining described previously. Incompetent perforating vein valves can also cause malleolar venules to become dilated and appear as fine red threads around the ankle. This condition, called ankle flair, is also diagnostic of a venous ulcer.

Medical hosiery is a conventional method of applying compression to normal shaped legs in order to prevent the development or recurrence of leg ulcers. However, these stockings are of limited value in the treatment of active ulceration, being difficult to apply over dressings. Compression bandages provide a slight advantage over medical hosiery. Compression bandages apply a pressure to the limb that is directly proportional to bandage tension but inversely proportional to the radius of curvature of the limb to which it is applied. This means that a bandage applied with constant tension to a limb of normal proportions will automatically produce graduated compression with the highest pressure at the ankle. This pressure will gradually reduce up the leg as the circumference increases.

As can be readily appreciated, it is cumbersome and difficult to apply uniform tension to the compression bandage as it is applied to the treated limb. Thus only highly skilled caregivers are able to effectively apply them. Moreover, once secured to the treated limb, care and attention must be given to ensure that the bandage does not slip or become displaced as this will lead to multiple layers forming, which in turn may lead to localized areas of high pressure, which can place the patient in direct risk of skin necrosis.

U.S. Pat. No. 7,276,037, filed Jan. 24, 2005, and U.S. Pat. No. 7,559,908, filed Apr. 20, 2005 are also directed towards compression apparatuses for applying localized pressure to the venous system of the leg and wound or ulcers respectively. The entire contents of these documents are incorporated herein by this reference.

The device of U.S. Pat. No. 7,276,037 includes a flexible member adapted to wrap around the extremity to secure an air bladder chamber to the extremity. An air pumping mechanism is operated to inflate the air bladder chamber to a pressurized state. One or more fluid-filled pressurized members are provided, each separate and distinct from the flexible member and the air bladder chamber and thus readily moveable relative to the flexible member and the air bladder chamber. The pressurized member is operably disposed between the extremity and the flexible member and applies increased localized pressure to the extremity during use. The pressurized member can be positioned during use such that it covers a venous ulcer (or other treatment site) and applies increased localized pressure to the treatment site in order to promote healing.

The compression apparatus of U.S. Pat. No. 7,559,908 also discloses includes a fluid-filled member, and further provides an associated sponge attached thereto. As shown in FIGS. 2F-2H, the sponge is retained by a flange, formed from the three walls of the fluid-filled member. The flange extends around the periphery of the apparatus, and in some embodiments, can be used to retain the sponge. The presence of the flange has a number of disadvantages. Because the flange is present around the entire peripheral edge, the flange material can irritate the patient, the wound, and the surrounding healthy tissue. In addition, the flange creates a non-therapeutic perimeter and increases the size of the corresponding dressing. This can limit the application of the apparatus at a treatment site, as the overall size of the unit can become too large to fit the treatment site.

In view of the foregoing, it can be seen that there are many problems, obstacles and challenges associated with the current treatments of leg ulcers. Thus, there is a need in the art to provide a device, for the treatment of venous ulcers or other wounds of the leg that is capable of being effectively self administered by a patient. Furthermore, it is desirable to have a device which produces a desired localized treatment, that does not severely limit the mobility of the patient or cause unwanted discomfort due to irritation or contamination to the patient's skin at the treatment site.

### SUMMARY OF THE INVENTION

The subject invention is directed to a device for providing therapeutic pressure to a treatment site. The devise comprises a bladder portion and a dressing portion. The bladder portion has opposed upper and lower exterior walls sealed to one another along common peripheral edges thereof to form an interior pressure chamber therebetween. The interior pressure chamber may be filled with a fluid selected from the group consisting of a gas, a liquid, and a gel.

An overall shape of the bladder portion may be selected from a number of geometric shapes including square, rectangle, circle, oval, triangle and crescent. In one embodiment, the upper and lower exterior walls are joined together in a sealed area towards a center of the interior pressure chamber. The size of the sealed area may vary depending on a size of the wound. A size and shape of the bladder portion may also vary and may be determined based on a size and type of the treatment site respectively.

The dressing portion has opposed upper and lower surfaces, wherein the upper surface of the dressing portion is attached to the lower exterior wall of the bladder portion at the peripheral edge of the bladder portion. In one embodiment, the dressing portion extends beyond the entire outer peripheral edge of the dressing portion. Because the bladder portion does not have a flange extending around the periphery of the device, the bladder portion does not contact the patient's skin. Rather, in use, the dressing portion is the only part of the device in contact with the treatment site. The dressing portion may be removable from the bladder portion. A material of the dressing portion may be selected from the group consisting of foam, impregnated foam, sponge, alginate and combinations thereof.

The device may further comprise a holder portion for positioning the dressing portion against the treatment site and securing around the limb so that the desired pressure is supplied to the treatment site. The holder may be detachable from the dressing portion or integrated therewith. The structure of the holder portion may be selected based on a location and type of treatment site, in order to maximize the comfort and effectiveness of the treatment for the patient.

In one embodiment, the holder portion is an adhesive strap attached to the bladder portion and wrapped around the limb such that no adhesive is in contact with the limb. In another embodiment, the holder portion is one or more adhesive tabs extending from the bladder portion. The configuration of the one or more adhesive tabs may be selected based on the location of the treatment site. In yet another embodiment, the holder portion is a covering provided over the entire bladder portion and attached to the limb using an adhesive. In another embodiment, the holder portion is a cloth bandage. In another embodiment, the holder portion is a strap having hook and loop fastening means provided therewith.

The subject invention is also directed to a device comprising:

a bladder portion having opposed upper and lower exterior walls sealed to one another along common peripheral edges thereof to form an interior pressure chamber therebetween, wherein the upper and lower exterior walls are joined together in a first sealed area along a perimeter of the common peripheral edges. The device also includes a dressing portion for contacting the treatment site, the dressing portion having opposed upper and lower surfaces, wherein the upper surface of the dressing portion is attached to the lower exterior wall of the bladder portion at the peripheral edge of the bladder portion. In one embodiment, the peripheral edge of the dressing portion extends beyond the common peripheral edges of the bladder portion.

In one embodiment, the upper and lower exterior walls are joined together in a second sealed area towards a center of the interior pressure chamber. The device may further be defined such that the height of the interior pressure chamber varies increasingly when measured from the first sealed area to the second sealed area. In this way, the chamber takes on a dome three-dimensional shape having a flattened surface at the peak of the dome. In one embodiment, the device further comprises a holder portion which may be detachable or integrated with the bladder portion. The holder portion may be integrated on the first sealed area. The holder portion is used to position the dressing portion against the treatment site of the limb. One skilled in the art would appreciate that the device may be further modified as described above.

These and other aspects, benefits and advantages of the devices of the subject invention will become more readily apparent from the following description taken in conjunction with the drawings.

### BRIEF DESCRIPTION OF THE DRAWINGS

So that those having ordinary skill in the art to which the subject invention pertains will more readily understand how to make and use the devices of the subject invention, preferred embodiments thereof will be described in detail herein below with reference to the drawings, wherein:

FIG. 1 is a perspective view of a device for providing pressure to a treatment site according to one embodiment of the subject invention, the device having an oval shape, showing the bladder portion having a sealed area towards a center of the interior pressure chamber;

FIG. 2 is a cross-sectional view taken along line 2-2 of the device of FIG. 1;

FIG. 3 is a perspective view of a device for providing pressure to a treatment site according to another embodiment of the subject invention, the device having a crescent shape;

FIG. 4 is a cross-sectional view, taken along line 4-4 of FIG. 3;

FIG. 5A is a perspective view of a device for providing pressure to a treatment site according to yet another embodiment of the subject invention, the device having a dome-shaped interior pressure chamber towards a center of the bladder portion;

FIG. 5B is a perspective view of a device for providing pressure to a treatment site according to yet another embodiment of the subject invention, the device having a dome-shaped interior pressure chamber towards a center of the bladder portion, the dressing portion extending beyond the peripheral edges of the bladder portion;

FIG. 6A is a cross-sectional view taken along line 6-6 of the device of FIG. 5A;

FIG. 6B is a cross-sectional view taken along line 6′-6′ of the device of FIG. 5B;

FIG. 7 is a perspective view of the device of FIG. 5A having a holder portion which includes a number of adhesive tabs provided on a first sealed area of the bladder portion;

FIG. 8 is perspective view of a device according to FIG. 1 where the treatment site is a malleolus of a foot, the device including adhesive (not shown) provided on the dressing portion such that the device is self-adhered to the patient's skin without the aid of a holder portion;

FIG. 9 is perspective view of a device according to FIG. 1 where the treatment site is a malleolus of a foot, and the device includes a holder portion which includes an adhesive strap attached to the bladder portion for securing the device into position; and

FIG. 10 is a perspective view of the device according to FIG. 5A having a holder portion consisting of a covering over the entire bladder and dressing portions.

### DETAILED DESCRIPTION OF THE PREFERRED EMBODIMENTS

Preferred embodiments of the subject invention are described below with reference to the accompanying drawings, in which like reference numerals represent the same or similar elements. One of ordinary skill in the art would appreciate that while the devices discussed herein are described in relation to compression therapy of treatment sites on the leg and foot, the scope of the invention is not limited to those exemplary applications and may be sized and shaped for the anatomical portion for which compression therapy is needed.

The subject invention provides compression to treatment sites on the extremities, including for example, the lower leg and foot. The subject invention provides devices that are simpler and less irritating to the patient's skin than current systems. In one embodiment, the subject invention provides a device that includes a dressing attached to a bladder that provides comfortable and uniform pressure to a wound when mechanical force is used. The devices provided herein are suitable for treatment of wounds such as surgical scars, burn scars, bedsores, and skin ulcers of all types. The devices may be used alone and attached to the skin using a holder. In addition, the devices of the subject invention may also be provided within a compression wrap in order to supply additional pressure to the treatment site. One exemplary wrap into which the devices of the subject invention may be incorporated is described in co-pending U.S. patent application Ser. No. 12/855,185 filed on Aug. 12, 2010. The entire contents of U.S. patent application Ser. No. 12/855,185 are incorporated herein by this reference.

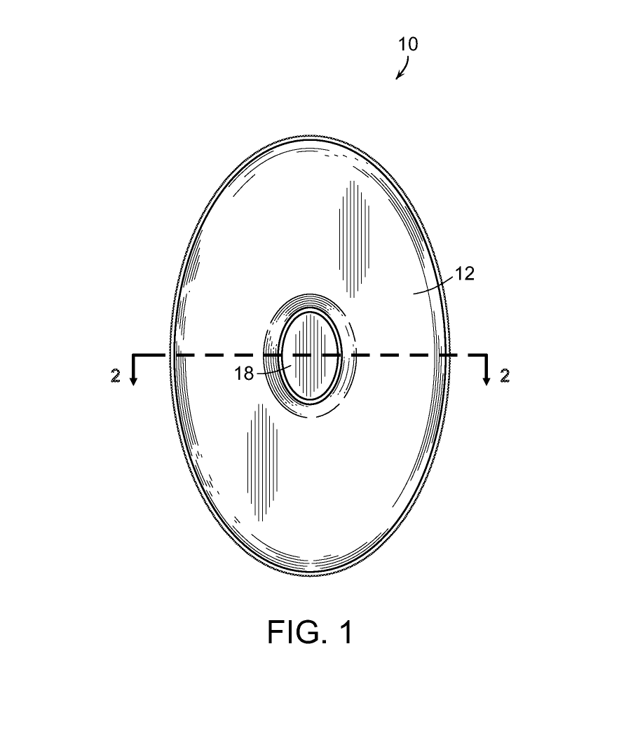

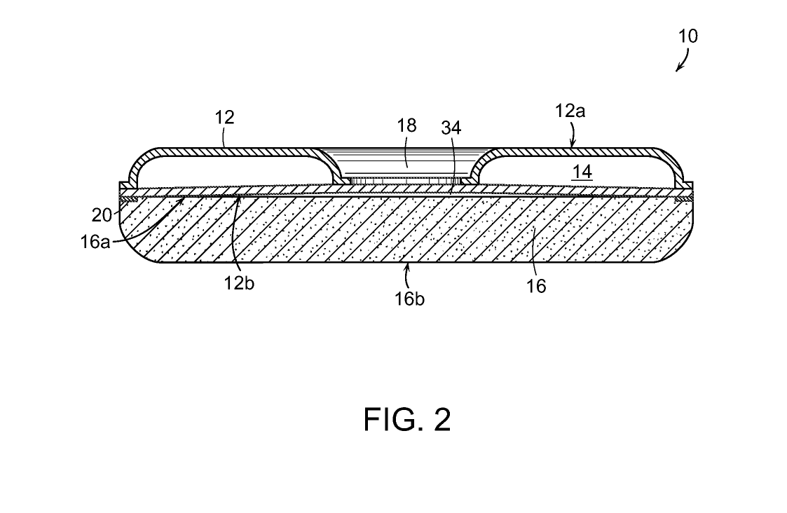

Referring now to FIGS. 1 and 2, one embodiment of a device for providing therapeutic pressure to a treatment site according to the subject invention is illustrated. The device 10 includes a bladder portion 12 which provides comfortable and uniform compression to a wound when mechanical force is used. The bladder portion 12 is comprised of two walls, upper exterior wall 12a and lower exterior wall 12b. The upper exterior wall 12a and the lower exterior wall 12b are opposed to each other and are sealed to one another along common peripheral edges thereof to form an interior pressure chamber 14 therebetween. The upper exterior wall 12a is configured with a substantially uniform height so that substantially uniform pressure is supplied to the wound. The interior pressure chamber 14 may be constructed with a predetermined amount of a contained gas, typically air. The contained gas amount may be less, equal or more than the unstressed volume of the interior pressure chamber 14 at ambient pressure. Other fluids such as liquids, gels and other gasses may also be used.

The dressing portion 16 is typically made of a porous membrane that has an upper surface 16a and a lower surface 16b. The dressing portion 16 may be made of foam, impregnated foam, sponge, alginate, or any known wound dressing material. The dressing portion 16 may also be trimmed at the outer edge, around the peripheral edge of the bladder portion 12, to provide a custom fit to a patient's wound. The dressing portion 16 is the only material in contact with the treatment site.

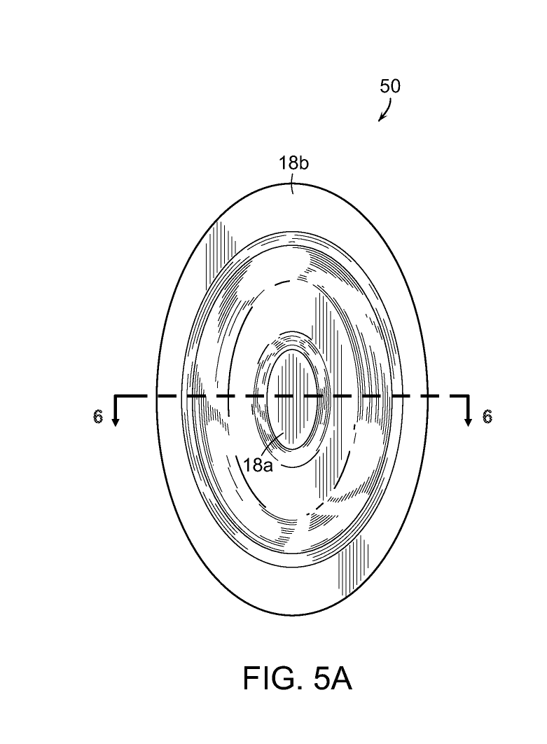

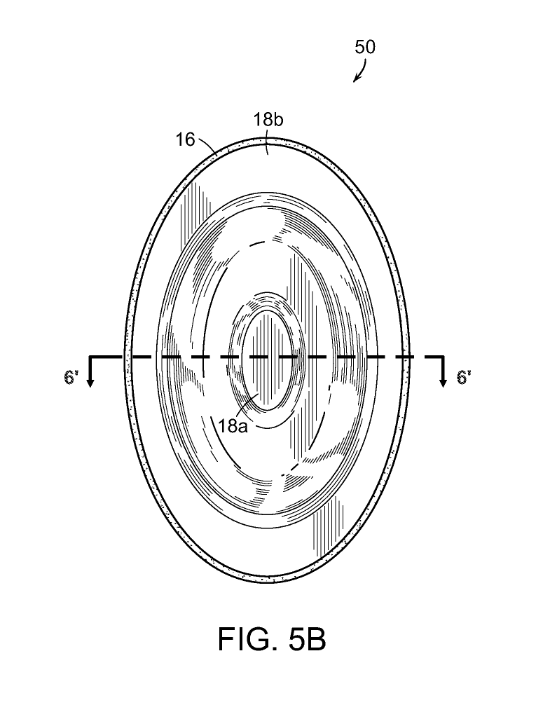

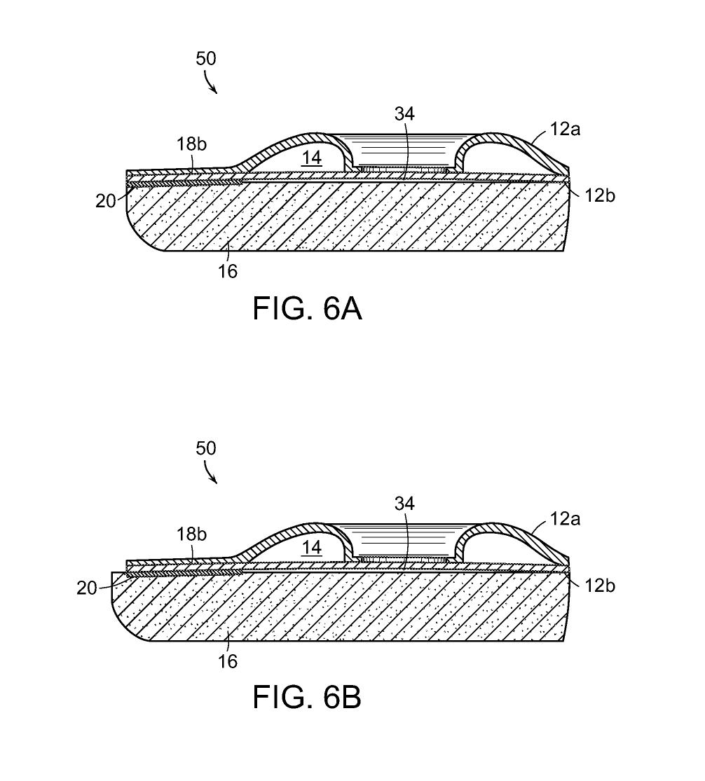

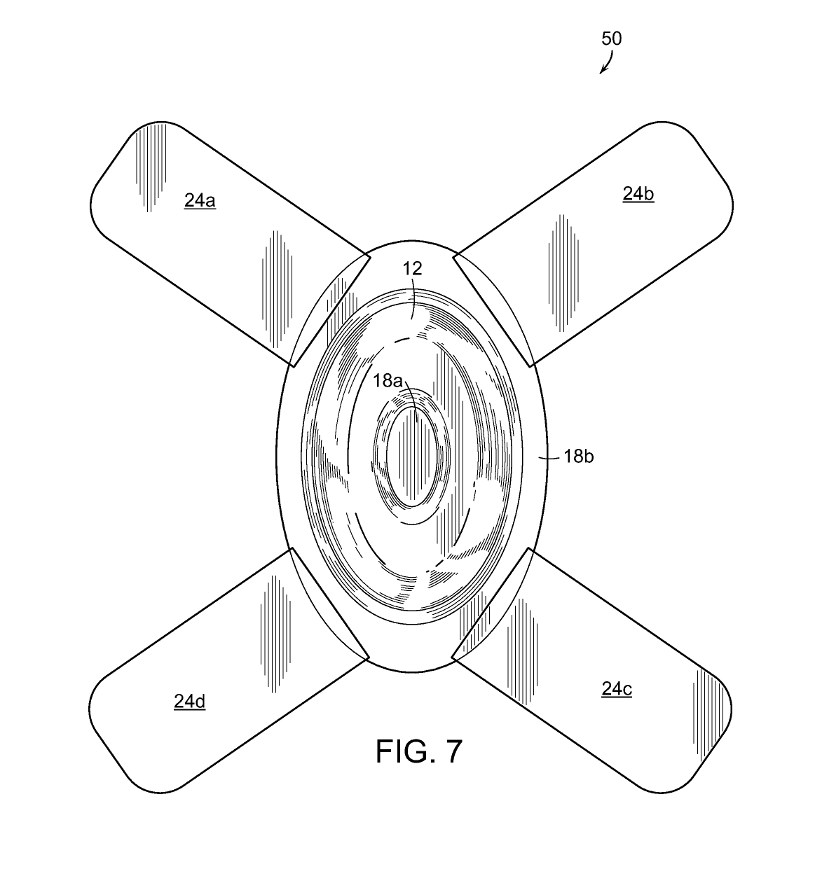

The dressing portion 16 and the bladder portion 12 are joined together such that the upper surface 16a of the dressing portion 16 is attached to the lower exterior wall 12b of the bladder portion 12 at the peripheral edge of the bladder portion 12. The two portions are attached such that there is a small void or space 34 between the dressing portion 16 and the bladder portion 12. When in use, the lower surface 16b of the dressing portion 16 is positioned in direct contact with the wound. The dressing portion 16 may be provided such that it is integrated with the bladder portion 12 and pre-attached during manufacture. Alternatively, the dressing portion 16 may be provided in detachable form, such that it can be adhered to the bladder portion 12 by an adhesive portion 20 at the peripheral edge of the bladder portion 12. The dressing portion 16 may be attached to the bladder portion 12 by a medical professional or by the patient at the time of use. As shown in FIGS. 5B and 6B, the dressing portion 16 may have a diameter that is greater than the bladder portion 12 such that it extends beyond the peripheral edge of the bladder portion 12. In this embodiment, the bladder portion 12 is joined to the dressing portion 16, for example, using an adhesive portion 20 at a small distance inward of the peripheral edge of the dressing portion 16. The dressing portion 16 may alternatively be joined to the bladder portion 12 using a welding process, as described herein.

The bladder portion 12 acts as a bolster to the dressing portion 16. Accordingly, the height of the bladder portion 12 can be varied by controlling an amount of fluid within the interior pressure chamber 14 in order to provide different levels of pressure to the wound. The device 10 of the subject invention may also be provided with a pressure gauge, such as a monometer, so that the amount of pressure supplied to the wound can be measured. The monometer may be provided in conjunction with a wrap which surrounds the entire device and relevant portion of the limb, for securing the device 10 in place.

A variety of processes may be employed to construct the devices of the subject invention. During manufacture, the two walls 12a, 12b of the bladder portion 12 are joined with the dressing portion 16. The walls 12a, 12b are typically made from materials comprising polyurethane films or other suitable plastics, are heated and fused to the dressing portion 16 by any number of processes including heat sealing, radio frequency welding, impulse welding, ultrasonic welding or other methods.

In one embodiment, impulse welding is used to join the bladder portion 12 to the dressing portion 16. During manufacture, the bladder portion 12 and the dressing portion 16 are heated and fused together by clamping them together in close contact with a shielded electric heating element. The welding process is carried out using pincer-like contact rails, in which the materials to be welded are placed between the contact rails and the necessary heat and pressure applied by closing the pincers. This process, unlike conventional assembly methods, enables the components to be welded together without creating a flange or lip around the peripheral edge of the dressing portion 16. This feature has the advantage of alleviating unwanted discomfort due to irritation or contamination of the patient's skin due to unwanted contact with the flange of the bladder portion 12 at the treatment site of conventional devices.

In contrast to conventional devices, the devices of the subject invention are adapted and configured to ensure that the dressing portion 16 is the only material in contact with the patient's wound. Due to the low clamping pressures required for impulse welding, and the melting and subsequent joining of the polymer materials being localized at their mating surfaces, this process prevents the permanent collapse and related reduction in absorbency of the porous or open celled structure of the dressing portion 16.

Ultrasonic welding is another assembly process that may be used to join the materials of the dressing portion 16 and the bladder portion 12, without creating a side flange or reducing the absorbency of the open celled structure of the dressing portion 16. During ultrasonic welding, the dressing portion 16 and the polymer materials of the bladder portion 12 are clamped under pressure between a horn and a fixed nest. The horn is then cycled perpendicular to the material surfaces to be joined at a prescribed frequency and amplitude driven by ultrasonic energy. Similar to impulse welding, ultrasonic welding produces a melt zone that is localized at the surfaces of the materials to be joined. Both impulse and ultrasonic welding may be employed to assemble the bladder portion 12 to the dressing portion 16, or to simply weld the upper exterior wall 12a and lower exterior wall 12b to form the bladder portion 12 of each of the device configurations provided herein.

Adhesive bonding is still another method of joining the bladder portion 12 to the dressing portion 16 without creating a patient side flange or reducing the absorbency of the open celled structure of the dressing portion 16. A suitable adhesive portion 20 is applied to one or both of the surfaces to be assembled, around the peripheral edge, and they are aligned and pressed together until attached, as shown in FIGS. 2, 4, 6A and 6B.

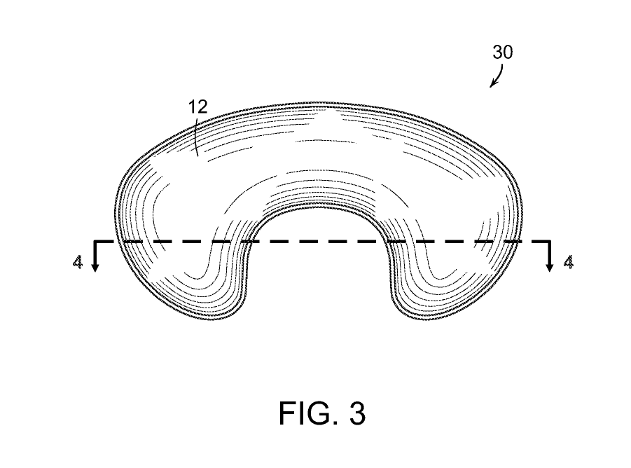

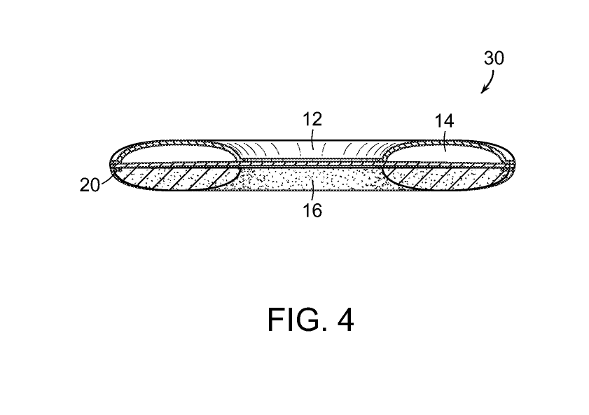

The overall shape of the devices of the subject invention may vary. For example, the device 10 of the subject invention is provided having an oval shape. However, in one embodiment, the device 30 may have a crescent shape as shown in FIGS. 3 and 4. One would readily appreciate that the devices of the subject invention could be configured in a number of geometric shapes, such as circular, square, rectangle, triangle etc. The devices of the subject invention can be adapted and configured to various shapes and sizes in order to ensure a best fit, such that the device conforms to both the wound and the anatomical shape of the treatment site.

The oval-shaped device 10 illustrated in FIG. 1 may be further configured such that the upper exterior wall 12a and lower exterior wall 12b of the bladder portion 12 are joined together in a sealed area 18 towards a center of the bladder portion 12. This creates a donut-shaped interior pressure chamber 14, as shown in FIG. 2. This donut-like configuration assures adequate pressure to the perimeter of the wound, which helps to draw moisture from the wound into the dressing portion 16, further aiding in the healing process. The donut-shaped configuration of the interior pressure chamber 14 also helps to pressure stimulate cell activity for healing. In addition, the donut-shaped configuration of the interior pressure chamber 14 also allows for adequate pressure to the outer wound when placed over a rigid anatomy such as the chin bone or malleolus.

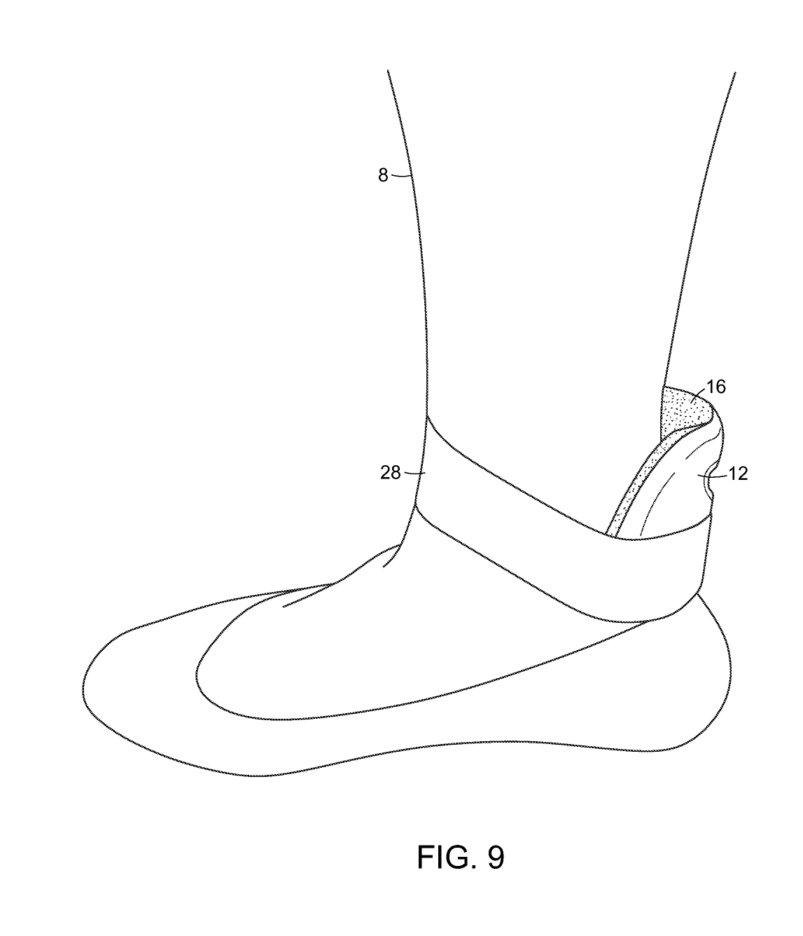

The donut-shape of the interior pressure chamber 14 is particularly useful for wounds on the malleolus of the leg 8 as shown in FIG. 8. The device 10 provides increased comfort to the patient by alleviating excess pressure to the malleolus. In this embodiment, the device 10 may be provided with am adhesive on the lower surface 16b of the dressing portion 16 so that the device 10 may be applied by the patient to the treatment site without the use of a holder portion.

Turning to FIG. 5A, another embodiment of the subject invention is illustrated. Device 50 also includes a bladder portion 12 and a dressing portion 16. The bladder portion 12 is characterized by upper exterior wall 12a and lower exterior wall 12b sealed to one another along common peripheral edges thereof to form an interior pressure chamber 14 therebetween. In this embodiment, the bladder portion 12 includes two sealed areas of the upper and lower exterior walls 12a, 12b. A first sealed area 18a is positioned towards a center of the interior pressure chamber 14. A second sealed area 18b is also present along a perimeter of the common peripheral edges. The width of the first sealed area 18a and the second sealed area 18b may vary based on the desired dimensions of the interior pressure chamber 14 defined between the two areas 18a, 18b.

FIG. 6A is a cross sectional view of the device 50 of FIG. 5A taken along line 6-6. FIG. 6A illustrates a feature of device 50 where a height of the interior pressure chamber 14 varies increasingly when measured from the second sealed area 18b to the first sealed area 18a, such that the largest height is measured near the first sealed area 18a. This variable height is achieved during the manufacture process, such that the bladder portion 12 has a three-dimensional dome shape. Again the interior pressure chamber 14 has a donut shape. In this embodiment, however, the device 50 provides a greater amount of pressure towards a center of the wound and gradually less pressure towards to the outer perimeter of the wound, in contrast to the device 10 of FIG. 1.

FIG. 5B is a perspective view of a device 50 for providing pressure to a treatment site according to yet another embodiment of the subject invention. FIG. 6B is the corresponding cross-sectional view of the device 50 of FIG. 5B taken along line 6′-6′. The device 50 illustrated in FIGS. 5B and 6B also has a dome-shaped interior pressure chamber 14 towards a center of the bladder portion 12. Here, the dressing portion 16 extends beyond the peripheral edges of the bladder portion 12, further ensuring that the dressing portion 16 is the only material in contact with the wound.

The dome-shape of the embodiment of the interior pressure chamber 14 of the bladder portion 12 illustrated in FIGS. 5A and 5B has a number of benefits and advantages. The dome-shape assures adequate pressure to a deep center of a patient's wound. In addition, the tapered sides at the outer edge of the interior pressure chamber 14 provides a gentle transition from the maximum pressure at the wound to a lower pressure surrounding the wound, thereby eliminating the abrupt transition at the perimeter of bladders of conventional devices.

One method of construction of the dome-shaped interior pressure chamber 14 is to vacuum thermoform the upper exterior wall 12a. In this process a polymer film is heated to an appropriate temperature below its melting point and drawn by a vacuum into a shaped cavity or over a shaped core and allowed to cool. The cavity is typically made of an appropriate rigid material such as epoxy, brass or steel with one or more vacuum holes provided to create a pressure differential between the two sides of the film and force the softened material against the shaped tooling. A shrink factor is applied to the tooling to compensate for material contraction during cooling. Once the plastic resin is allowed to sufficiently cool it may be removed from the tool and will retain its vacuum thermoformed dome shape. This can then be assembled to the lower exterior wall 12b by one of the methods previously discussed.

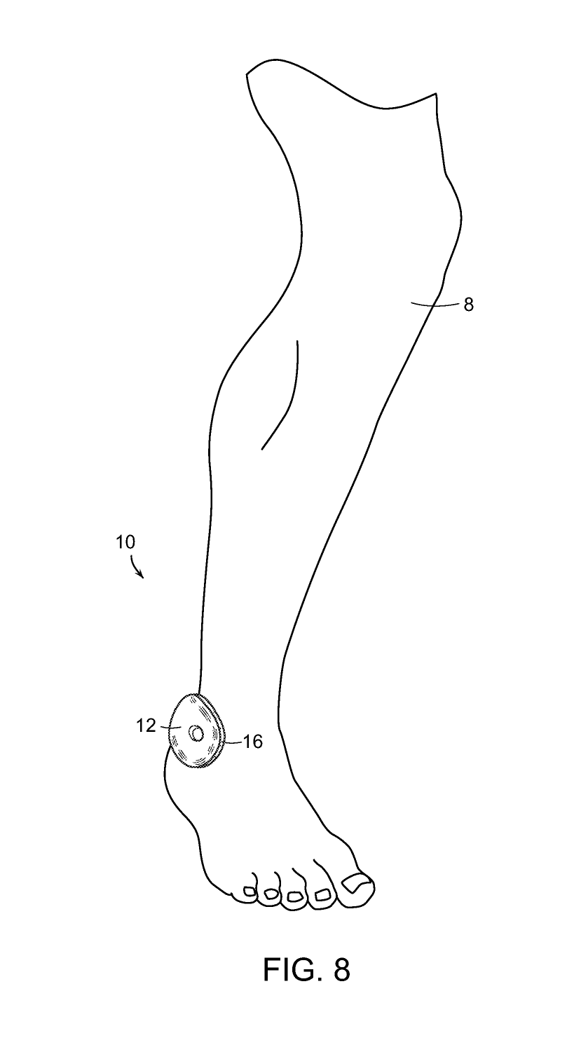

The subjection invention also provides for an optional holder portion which may be used in conjunction with each of the device configurations provided herein. Turning to FIG. 7, device 50, for example, may be provided with a holder portion integrated with the bladder portion 12 consisting of one or more adhesive tabs 24a, 24b, 24c, 24d. The adhesive tabs 24a-24d may be provided such that they are configured to be integrated with the second sealed area 18b, and extend therefrom. Alternatively, one or more of the adhesive tabs 24a-24d may be attached by the patient. In this embodiment, the positioning and configuration of the adhesive tabs 24a-24d may be selected based on the dimensions and anatomical considerations of the treatment site.

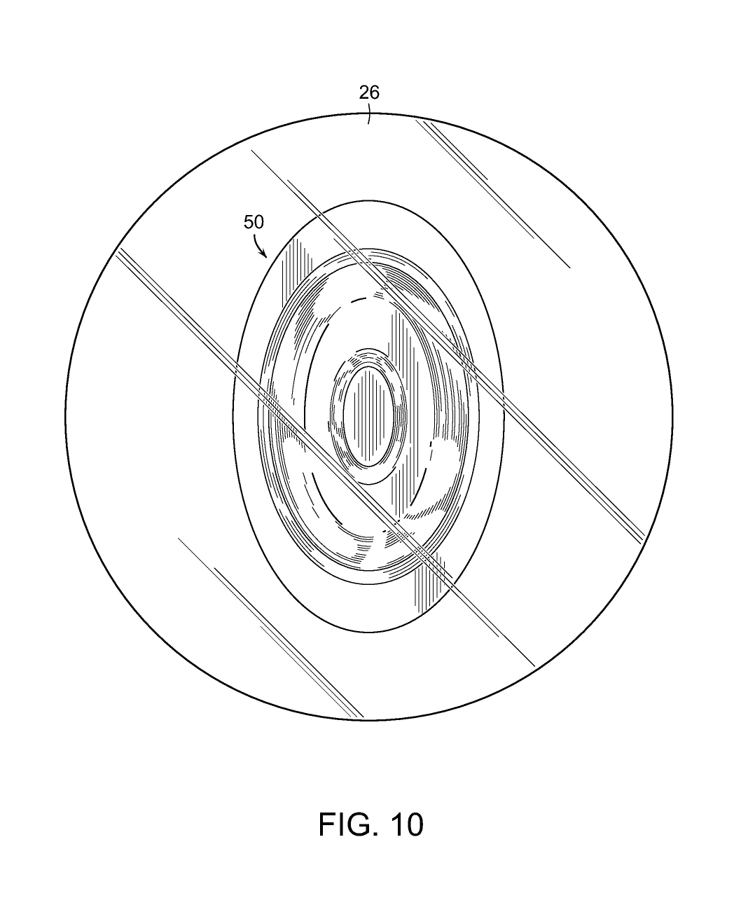

Turning to FIG. 9, the holder portion may also be provided as an adhesive strap 28 attached to the bladder portion 12 and wrapped around the leg 8 such that no adhesive is in contact with the limb 8. The holder portion may consist of a strap (not shown) having hook and loop fastening means, buckles or the like provided therewith in order to secure the device 50 to the treatment site and provide additional pressure to the wound in contact with the dressing portion 16. FIG. 10 illustrates yet another embodiment of a holder portion according to the subject invention. Here, the holder portion is an adhesive covering 26 provided over the entire device 50 in order to secure it to the limb 8. Other known means such as a cloth bandages and medical tape may also be used as a holder portion in connection with the devices of the subject invention.

While the subject invention of the present disclosure has been described with respect to preferred and exemplary embodiments, those skilled in the art will readily appreciate that various changes and/or modifications can be made to the invention without departing from the spirit or scope of the invention as described herein.

What is claimed is:

1. A device for providing therapeutic pressure to a treatment site comprising:
a) a bladder portion having opposed upper and lower exterior walls sealed to one another along common peripheral edges thereof to form an interior pressure chamber therebetween; and
b) a dressing portion for contacting the treatment site, the dressing portion having opposed upper and lower surfaces, wherein the upper surface of the dressing portion is attached to the lower exterior wall of the bladder portion at the peripheral edge thereof.

2. The device of claim 1, wherein the interior pressure chamber is filled with a fluid selected from the group consisting of a gas, a liquid, and a gel.

3. The device of claim 1, wherein a material of the dressing portion is selected from the group consisting of foam, impregnated foam, sponge, alginate and combinations thereof.

4. The device of claim 1, wherein an overall shape of the bladder portion is selected from the group consisting of a square, a rectangle, a circle, an oval, a triangle and a crescent.

5. The device of claim 1, wherein the upper and lower exterior walls are joined together in a sealed area towards a center of the interior pressure chamber.

6. The device of claim 1, wherein a size and shape of the bladder portion is determined based on a size and type of the treatment site respectively.

7. The device of claim 1, wherein the dressing portion is removable from the bladder portion.

8. The device of claim 1, further comprising a holder portion for positioning the dressing portion against the treatment site of the limb.

9. The device of claim 8, wherein the holder portion is an adhesive strap attached to the bladder portion and wrapped around the limb such that no adhesive is in contact with the limb.

10. The device of claim 8, wherein the holder portion is one or more adhesive tabs extending from the bladder portion.

11. The device of claim 10, wherein a configuration of the one or more adhesive tabs is selected based on the treatment site.

12. The device of claim 8, wherein the holder portion is a covering provided over the entire bladder portion and attached to the limb using an adhesive.

13. The device of claim 8, wherein the holder portion is a cloth bandage or a strap having hook and loop fastening means provided therewith.

14. The device of claim 1, wherein a peripheral edge of the dressing portion extends beyond the common peripheral edges of the bladder portion.

15. A device for providing therapeutic pressure to a treatment site comprising:
a) a bladder portion having opposed upper and lower exterior walls sealed to one another along common peripheral edges thereof to form an interior pressure chamber therebetween, wherein the upper and lower exterior walls are joined together in a first sealed area along a perimeter of the common peripheral edges; and
b) a dressing portion for contacting the treatment site, the dressing portion having opposed upper and lower surfaces, wherein the upper surface of the dressing portion is attached to the lower exterior wall of the bladder portion at the peripheral edge thereof.

16. The device of claim 15, wherein the upper and lower exterior walls are joined together in a second sealed area towards a center of the interior pressure chamber.

17. The device of claim 16, wherein a height of the interior pressure chamber varies increasingly when measured from the first sealed area to the second sealed area.

18. The device of claim 15, further comprising a holder portion integrated with the bladder portion on the first sealed area for positioning the dressing portion against the treatment site of the limb.

19. The device of claim 18, wherein the holder portion is one or more adhesive tabs extending from the bladder portion.

20. The device of claim 15, wherein the dressing portion extends beyond the common peripheral edges of the bladder portion.
